(19) United States
(12) Patent Application Publication
(10) Pub. No.: US 2022/0313575 A1
(43) Pub. Date: Oct. 6, 2022

(54) PRESERVATIVE SYSTEM

(71) Applicant: L'OREAL, Paris (FR)

(72) Inventors: Michele Nicole CRISTIANO, Old Bridge, NJ (US); Noah Louis WIEDER, Linden, NJ (US); Mark ZAW, Denville, NJ (US)

(73) Assignee: L'OREAL, Paris (FR)

(21) Appl. No.: 17/218,797

(22) Filed: Mar. 31, 2021

(51) Int. Cl. A61K 8/368; A61K 8/06; A61K 8/73; A61K 8/27

(52) U.S. Cl. CPC A61K 8/368; A61K 8/062; A61K 8/731; A61K 8/732; A61K 8/064; A61K 8/27; A61K 2800/10; A61K 2800/524

(57) ABSTRACT

A preservative system that includes salicylic acid; weak organic acid or its salts; and one or more divalent transition metal salts; and cosmetic compositions formulated with the preservative systems.

### FIELD OF THE INVENTION

The instant disclosure relates to preservative systems for keratinous tissue, particularly skin, that include salicylic acid, benzoic acid or its salts and divalent transition metal salts.

### BACKGROUND OF THE INVENTION

Preservatives are commonly used in personal care products. Preservatives are aimed at protecting such products from decay or spoilage, mainly caused by microorganisms. They typically possess anti-microbial activity.

Because preservative agents may cause adverse effects such as allergic responses and skin irritation, it is desirable to use them in the smallest amount possible in the cosmetics or other personal care products. Thus, a balance must be achieved by having an effective anti-microbial amount of the preservative or preservatives in the product, yet having that amount be as small as possible to avoid or reduce the chance of adverse effects. Also, it is desirable that the preservative or blend of preservatives be effective against the widest possible types of potentially harmful microorganisms that could cause decay or spoilage of personal care products. Furthermore, such preservatives should be chemically and physically compatible with the other ingredients in the personal care product.

### SUMMARY OF THE DISCLOSURE

The instant disclosure relates to preservative systems that are unique in comparison to well-known preservative systems because they aim to preserve highly natural formulas without the use of petrochemicals or alcohol to be suitable for all products formats including a jar packaging, which requires the most stringent level of challenge testing due to the product being exposed to the most contact with the environment. While not wishing to be bound by any particular theory, the inventors believe that the combination of salicylic acid, benzoic acid and one or more divalent metal salts contribute to the preservation of highly natural cosmetic systems. The preservative system typically includes:
a. salicylic acid;
b. weak organic acid or its salts;
c. of one or more divalent transition metal salts.

In some embodiments, the weak organic acid or its salts is chosen from benzoic acid, sorbic acid, levulinic acid, anisic acid, dehydroacetic acid, propionic acid and combinations thereof. In one embodiment, the weak organic acid or its salts is benzoic acid or its salts.

In some embodiments, the preservative system includes one or more divalent metal salts are chosen from a divalent metal salt of an organic acid, a divalent metal salt of an inorganic acid, and combinations thereof.

In one or more embodiments, the one or more divalent metal salts are chosen from salts of zinc, nickel, copper, silver, manganese, cobalt, and combinations thereof.

In some embodiments, the one or more divalent metal salts include divalent metal salts of an organic acid chosen from zinc lactate, zinc gluconate, zinc phenolsulfonate, zinc salicylate, zinc pyrrolidone carboxylic acid (Zinc PCA), zinc citrate, zinc ascorbate, copper gluconate, copper sulfate, their salt derivatives, and combinations thereof.

In some embodiments, the one or more divalent metal salts include divalent metal salts of an inorganic acid chosen from metal halides, metal hydroxides, metal sulfates, metal oxides, and combinations thereof. In one embodiments, the one or more divalent metal salts of an inorganic acid are chosen from salts of zinc, nickel, copper, silver, manganese, cobalt, and combinations thereof.

In some embodiments, the one or more divalent metal salts of an organic acid includes a zinc salt. In one embodiment, the zinc salt is zinc gluconate.

In some different aspect of the disclosure, some embodiments may include a cosmetic composition comprising the preservative system described in the instant disclosure, a suitable carrier system, and additives selected from fatty compounds, fillers, polymers, surfactants, solvents, essential oils, vegetal extract, conditioners, emollients, skin caring ingredients, thickeners, anti-oxidants, other preservatives, active ingredients, fragrances and combination thereof.

In one or more embodiments, the one or more divalent metal salts is present from about 0.01 to about 0.5 wt.% based on the total weight of the cosmetic composition.

In one or more embodiments, the salicylic acid is present from about 0.01 to about 0.5 wt.% based on the total weight of the cosmetic composition.

In some embodiments, the benzoic acid is present from about 0.01 to about 0.3 wt.% based on the total weight of the cosmetic composition.

In one or more embodiments, the cosmetic composition is in the form of an oil-in-water emulsion. In one embodiment, the cosmetic composition is in the form of a water-in-oil emulsion.

In one or more embodiments, the preservative systems describe in the instant disclosure may be used in hair compositions, skin care compositions, cleansing compositions, makeup compositions.

The preservative system of the instant disclosure contains salicylic acid, benzoic acid, one or more divalent transition metal salts and can be added to other cosmetic compositions. The cosmetic compositions may contain solvents or any of the other components mentioned hereinafter as components that can be added to the compositions according to the instant disclosure.

The compositions of the instant disclosure may be prepared by mixing the salicylic acid, the benzoic acid and the divalent metal salts. Solvent may be added after mixing or the components are mixed while being present in a solvent. Other components may be added during the mixing or afterwards. The said salicylic acid, benzoic acid, divalent metal salts may also be added to a premix of other components.

In one or more embodiments, a cosmetic composition may include a preservative system comprising salicylic acid, benzoic acid, one or more divalent transition metal salts and some additives selected from fatty compounds, fillers, surfactants, solvents, essential oils, vegetal extract, conditioners, emollients, skin caring ingredients, thickeners, anti-oxidants, other preservatives, active ingredients, fragrances and combination thereof.

The novel association identified in the preservative system and described in the instant disclosure may be applied in any cosmetic products, in particular highly natural formulas. The highly natural formulas can be effectively preserved for all product formats and highly water-based systems and emulsions. Cosmetic compositions comprising the novel preservative system that are appropriate for topical application to the keratinous tissue, such as skin, include composition in the form of a lotion, serum, gel, milk, liquid foundation or cream.

Suitable cosmetic system which include the novel preservative system include lotion or serum type of emulsions with a liquid or semi-liquid consistency of the milk type, obtained by dispersion of a fatty phase in an aqueous phase (O/W) or vice versa (W/O), including those employing silicone and non-silicone oils, or of suspensions or emulsions with a soft consistency of the aqueous gel or cream type, or of microcapsules or microparticles, or of vesicular dispersions of ionic and/or nonionic type or of foams, or they may be provided in the form of toners or micellar water, or they may be biphasic and include water and other solvents such as alcohols and other water-soluble solvents and oils that are not water miscible. Biphasic compositions require pre-application agitation to form an emulsion prior to application to the keratinous tissue, such as skin, which emulsion is of sufficient quality and stability to allow a uniform application of the emulsified phases. Thus, a suitable carrier system according to the disclosure may be selected from any one of oil-in-water emulsions, water-in-oil emulsions, silicone in water emulsions, dispersions, micellar waters, biphasic compositions, and foams.

In one or more embodiments, the cosmetic composition can be in the form of an oil-in-water emulsion. In some embodiments, the cosmetic composition is in the form of a water-in-oil emulsion. In some embodiments, the cosmetic composition is an aqueous system, aqueous serum, gel, light lotion, shampoo.

Another aspect of the instant disclosure is compositions having a preservative system comprising:
a. from 0.01 to 0.5 wt.% of salicylic acid;
b. from 0.01 to 0.3 wt.% of benzoic acid or its salts;
c. one or more divalent transition metal salts.

In some embodiments, some compositions may include:
A) A preservative system comprising:
   a. from 0.01 to 0.5 wt.% of salicylic acid;
   b. from 0.01 to 0.3 wt.% of benzoic acid or its salts;
   c. from about 0.01 to about 0.5 wt.% of one or more divalent transition metal salts;
B) from about 0.1 to about 15 wt.% of one or more fatty compound;
C) from about 0.1 to about 5 wt.% of one or more fillers;
D) from about 0.1 to about 10 wt.% of one or more surfactants; and
E) a suitable carrier.

In another exemplary embodiment, the instant disclosure is related to a method of preserving a composition by incorporating the preservative system described in the instant disclosure.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

It is to be understood that both the foregoing general description and the following detailed description are exemplary and explanatory only and are not restrictive of the disclosure.

### DETAILED DESCRIPTION OF THE DISCLOSURE

The instant disclosure relates to preservative systems that are unique in comparison to well-known preservative systems because they aim to preserve highly natural formulas without the use of petrochemicals or alcohol to be suitable for all products formats including a jar packaging, which requires the most stringent level of challenge testing due to the product being exposed to the most contact with the environment. The preservative system typically includes:
a. salicylic acid;
b. weak organic acid or its salts;
c. of one or more divalent transition metal salts.

The term “preservative system” or “preservative” used herein is understood to refer to preservative compositions that include ingredients that have both anti-microbial and anti-oxidant effects that can be incorporated into cosmetic and personal care formulations, thus would enhance the shelf life of such cosmetic and personal care formulations.

The term “solubility” used herein is understood to explain the ability to dissolve into a homogeneous solution with no change in visual or microscope cloudiness or particulates.

### SALICYLIC ACID

In accordance with the various embodiments, the preservative system according to the disclosure comprises salicylic acid.

Salicylic acid (from Latin salix, willow tree) is a lipophilic monohydroxybenzoic acid, a type of phenolic acid, and a beta hydroxy acid (BHA). It has the formula C7H603. This colorless crystalline organic acid is widely used in organic synthesis and functions as a plant hormone.

In accordance with the various embodiments, the amount of salicylic acid present in the preservative system is in a range from about 1.5 to about 95 wt.%, or from about 2.0 to about 94.5 wt.%, or from about 4.0 to about 91 wt.%, or from about 5.0 to about 90wt.%, or from about 7.0 to about 89% wt.%, from about 10 to about 80% wt.%, from about 15 to about 75 wt.% or any suitable combination, sub-combination, range, or sub-range thereof by weight, based on the weight of the preservative system.

Thus, any one of or a combination of salicylic acid is present, by weight, based on the total weight of the preservative system, from about 1.5, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, 10.0, 12.0. 14.0, 16.0, 18.0, 20.0, 22.0, 24.0, 26.0, 28.0, 30.0, 32.0, 34.0, 36.0, 38.0, 40.0, 42.0, 44.0, 46.0, 48.0 to about 48.0, 50.0, 52.0, 54.0, 56.0, 58.0, 60.0, 62.0, 64.0, 66.0, 68.0, 70.0, 72.0, 74.0, 76.0, 78.0, 80.0, 82.0, 84.0, 86.0, 88.0, 90.0, 92.9, 92.0, 93.0, 94.0, or 95.0% including increments and ranges therein and there between.

In accordance with the various embodiments, the amount of salicylic acid present in a cosmetic composition is in a range from about 0.01 to about 0.5 wt.%, or from about 0.05 to about 0.48 wt.%, or from about 0.1 to about 0.46 wt.%, or from 0.15 to about 0.4 wt%, or any suitable combination, sub-combination, range, or sub-range thereof by weight, based on the weight of the composition.

Thus, any one of or a combination of salicylic acid is present, by weight, based on the total weight of the cosmetic composition, from about 0.01, 0.02, 0.03, 0.04, 0.05, 0.06, 0.07, 0.08, 0.09, 0.10, 0.11, 0.12, 0.13, 0.14, 0.15 to about 0.15, 0.16, 0.17, 0.18, 0.19, 0.20, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49 or 0.5 wt.% including increments and ranges therein and there between.

### WEAK ORGANIC ACIDS

In accordance with the various embodiments, the preservative system according to the disclosure comprises weak organic acid or its salts.

In accordance with the various embodiments, the amount of weak organic acids or its salts present in the preservative system is in a range from about 1.5 to about 95 wt.%, or from about 2.0 to about 94.5 wt.%, or from about 4.0 to about 91 wt.%, or from about 5.0 to about 90 wt.%, or from about 7.0 to about 89% wt.%, from about 10 to about 80% wt.%, from about 15 to about 75 wt.% or any suitable combination, sub-combination, range, or sub-range thereof by weight, based on the weight of the preservative system.

Thus, any one of or a combination of weak organic acid or its salts is present, by weight, based on the total weight of the preservative system, from about 1.5, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, 10.0, 12.0. 14.0, 16.0, 18.0, 20.0, 22.0, 24.0, 26.0, 28.0, 30.0, 32.0, 34.0, 36.0, 38.0, 40.0, 42.0, 44.0, 46.0, 48.0 to about 48.0, 50.0, 52.0, 54.0, 56.0, 58.0, 60.0, 62.0, 64.0, 66.0, 68.0, 70.0, 72.0, 74.0, 76.0, 78.0, 80.0, 82.0, 84.0, 86.0, 88.0, 90.0, 92.9, 92.0, 93.0, 94.0, or 95.0% including increments and ranges therein and there between.

The weak organic acid may be chosen from benzoic acid, sorbic acid, levulinic acid, anisic acid, dehydroacetic acid, propionic acid and combinations thereof.

In accordance with the various embodiments, the amount of benzoic acid present in the preservative system is in a range from about 1.5 to about 95 wt.%, or from about 2.0 to about 94.5 wt.%, or from about 4.0 to about 91 wt.%, or from about 5.0 to about 90 wt.%, or from about 7.0 to about 89% wt.%, from about 10 to about 80% wt.%, from about 15 to about 75 wt.% or any suitable combination, sub-combination, range, or sub-range thereof by weight, based on the weight of the preservative system.

Thus, any one of or a combination of benzoic acid is present, by weight, based on the total weight of the preservative system, from about 1.5, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, 10.0, 12.0. 14.0, 16.0, 18.0, 20.0, 22.0, 24.0, 26.0, 28.0, 30.0, 32.0, 34.0, 36.0, 38.0, 40.0, 42.0, 44.0, 46.0, 48.0 to about 48.0, 50.0, 52.0, 54.0, 56.0, 58.0, 60.0, 62.0, 64.0, 66.0, 68.0, 70.0, 72.0, 74.0, 76.0, 78.0, 80.0, 82.0, 84.0, 86.0, 88.0, 90.0, 92.9, 92.0, 93.0, 94.0, or 95.0% including increments and ranges therein and there between.

In accordance with the various embodiments, the amount of weak organic acid present in a cosmetic composition is in a range from about 0.01 to about 0.3 wt.%, or from about 0.05 to about 0.28 wt.%, or from about 0.1 to about 0.2 wt.%, or from 0.15 to about 0.24 wt.%, or any suitable combination, sub-combination, range, or sub-range thereof by weight, based on the weight of the composition.

Thus, any one of or a combination of weak organic acid is present, by weight, based on the total weight of the cosmetic composition, from about 0.01, 0.02, 0.03, 0.04, 0.05, 0.06, 0.07, 0.08, 0.09, 0.10, 0.11, 0.12, 0.13, 0.14, 0.15 to about 0.15, 0.16, 0.17, 0.18, 0.19, 0.20, 0.21, 0.22, 0.23, 0.24, 0.25, 0.26, 0.27, 0.28, 0.29, or 0.3 wt.% including increments and ranges therein and there between.

### DIVALENT TRANSITION METAL SALTS

In accordance with the various embodiments, the amount of divalent transition metal salts present in the preservative system is in a range from about 1.5 to about 95 wt.%, or from about 2.0 to about 94.5 wt.%, or from about 4.0 to about 91 wt.%, or from about 5.0 to about 90 wt.%, or from about 7.0 to about 89% wt.%, from about 10 to about 80% wt.%, from about 15 to about 75 wt.% or any suitable combination, sub-combination, range, or sub-range thereof by weight, based on the weight of the preservative system.

Thus, any one of or a combination of divalent transition metal salts is present, by weight, based on the total weight of the preservative system, from about 1.5, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, 10.0, 12.0. 14.0, 16.0, 18.0, 20.0, 22.0, 24.0, 26.0, 28.0, 30.0, 32.0, 34.0, 36.0, 38.0, 40.0, 42.0, 44.0, 46.0, 48.0 to about 48.0, 50.0, 52.0, 54.0, 56.0, 58.0, 60.0, 62.0, 64.0, 66.0, 68.0, 70.0, 72.0, 74.0, 76.0, 78.0, 80.0, 82.0, 84.0, 86.0, 88.0, 90.0, 92.9, 92.0, 93.0, 94.0, or 95.0% including increments and ranges therein and there between.

In accordance with the various embodiments, the amount of divalent metal salts present in a cosmetic composition is in a range from about 0.01 to about 0.5 wt.%, or from about 0.05 to about 0.48 wt.%, or from about 0.1 to about 0.46 wt.%, or from 0.15 to about 0.44 wt%, or any suitable combination, sub-combination, range, or sub-range thereof by weight, based on the weight of the composition.

Thus, any one of or a combination of divalent metal salts is present, by weight, based on the total weight of the cosmetic composition, from about 0.01, 0.02, 0.03, 0.04, 0.05, 0.06, 0.07, 0.08, 0.09, 0.10, 0.11, 0.12, 0.13, 0.14, 0.15 to about 0.15, 0.16, 0.17, 0.18, 0.19, 0.20, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49 or 0.5 wt.% including increments and ranges therein and there between.

### Divalent Metal Salts of an Inorganic Acid

The one or more divalent metal salts of an inorganic acid may be chosen from metal halides, metal hydroxides, metal sulfates, metal oxides, and combinations thereof.

Suitable examples of the one or more divalent metal salts of an inorganic acid may be chosen from zinc chloride, zinc sulfate, zinc nitrate, zinc carbonate and hydrogen carbonate, zinc phosphate, and combinations thereof.

In accordance with the various embodiments, the amount of divalent metal salts of an inorganic acid present in the preservative system is in a range from about 1.5 to about 95 wt.%, or from about 2.0 to about 94.5 wt.%, or from about 4.0 to about 91 wt.%, or from about 5.0 to about 90 wt.%, or from about 7.0 to about 89% wt.%, from about 10 to about 80% wt.%, from about 15 to about 75 wt.% or any suitable combination, sub-combination, range, or sub-range thereof by weight, based on the weight of the preservative system.

Thus, any one of or a combination of divalent transition metal salts of an inorganic acid is present, by weight, based on the total weight of the preservative system, from about 1.5, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, 10.0, 12.0. 14.0, 16.0, 18.0, 20.0, 22.0, 24.0, 26.0, 28.0, 30.0, 32.0, 34.0, 36.0, 38.0, 40.0, 42.0, 44.0, 46.0, 48.0 to about 48.0, 50.0, 52.0, 54.0, 56.0, 58.0, 60.0, 62.0, 64.0, 66.0, 68.0, 70.0, 72.0, 74.0, 76.0, 78.0, 80.0, 82.0, 84.0, 86.0, 88.0, 90.0, 92.9, 92.0, 93.0, 94.0, or 95.0% including increments and ranges therein and there between.

In accordance with the various embodiments, the amount of divalent metal salts of an inorganic acid present in a cosmetic composition is in a range from about 0.01 to about 0.5 wt.%, or from about 0.05 to about 0.48 wt.%, or from about 0.1 to about 0.46wt.%, or from 0.15 to about 0.44 wt%, or any suitable combination, sub-combination, range, or sub-range thereof by weight, based on the weight of the composition.

Thus, any one of or a combination of divalent metal salts of an inorganic acid is present, by weight, based on the total weight of the cosmetic composition, from about 0.01, 0.02, 0.03, 0.04, 0.05, 0.06, 0.07, 0.08, 0.09, 0.10, 0.11, 0.12, 0.13, 0.14, 0.15 to about 0.15, 0.16, 0.17, 0.18, 0.19, 0.20, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 wt.% including increments and ranges therein and there between.

### Divalent Metal Salts of an Organic Acid

The one or more divalent metal salts of an organic acid may be chosen from metal salts wherein the organic acid is chosen from Cl to C9 monocarboxylic or di-carboxylic acids, polycarboxylic acids, hydroxy-carboxylic acids, and fatty acids having at least 10 carbon atoms including ascorbic acid, formic acid, acetic acid, glycolic acid, gluconic acid, lactic acid, mandelic acid, oxalic acid, maleic acid, malonic acid, glyoxylic acid, succinic acid, adipic acid, fumaric acid, sebacic acid, including citric acid, tartaric acid, malic acid, 2-hydroxy malonic acid, 2-hydroxyl n-butyl 1,2,4-tricarboxylic acid, glutaric acid, glucaric acid, 2-hydroxy n-butyl 1,3,4-tricarboxylic acid, pyrrolidone carboxylic acid, and phenolsulfonic acid.

In an embodiment, the one or more divalent metal salts of an organic acid are chosen from zinc lactate, zinc gluconate, zinc phenolsulfonate, zinc salicylate, zinc pyrrolidone carboxylic acid (Zinc PCA), zinc ascorbate, zinc citrate, their salt derivatives thereof, and combinations thereof.

In an embodiment, the one or more divalent metal salts of an organic acid includes a zinc salt.

In an embodiment, the zinc salt is zinc gluconate.

In accordance with the various embodiments, the amount of divalent metal salts of an organic acid present in the preservative system is in a range from about 1.5 to about 95 wt.%, or from about 2.0 to about 94.5 wt.%, or from about 4.0 to about 91 wt.%, or from about 5.0 to about 90 wt.%, or from about 7.0 to about 89% wt.%, from about 10 to about 80% wt.%, from about 15 to about 75 wt.% or any suitable combination, sub-combination, range, or sub-range thereof by weight, based on the weight of the preservative system.

Thus, any one of or a combination of divalent transition metal salts of an organic acid is present, by weight, based on the total weight of the preservative system, from about 1.5, 1.8, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.8, 7.0, 7.2, 7.4, 7.6, 7.8, 8.0, 8.2, 8.4, 8.6, 8.8, 9.0, 9.2, 9.4, 9.6, 9.8, 10.0, 12.0. 14.0, 16.0, 18.0, 20.0, 22.0, 24.0, 26.0, 28.0, 30.0, 32.0, 34.0, 36.0, 38.0, 40.0, 42.0, 44.0, 46.0, 48.0 to about 48.0, 50.0, 52.0, 54.0, 56.0, 58.0, 60.0, 62.0, 64.0, 66.0, 68.0, 70.0, 72.0, 74.0, 76.0, 78.0, 80.0, 82.0, 84.0, 86.0, 88.0, 90.0, 92.9, 92.0, 93.0, 94.0, or 95.0% including increments and ranges therein and there between.

In accordance with the various embodiments, the amount of divalent metal salts of an organic acid present in a cosmetic composition is in a range from about 0.01 to about 0.5 wt.%, or from about 0.05 to about 0.48 wt.%, or from about 0.1 to about 0.46 wt.%, or from 0.15 to about 0.44 wt%, or any suitable combination, sub-combination, range, or sub-range thereof by weight, based on the weight of the composition.

Thus, any one of or a combination of divalent metal salts of an organic acid is present, by weight, based on the total weight of the cosmetic composition, from about 0.01, 0.02, 0.03, 0.04, 0.05, 0.06, 0.07, 0.08, 0.09, 0.10, 0.11, 0.12, 0.13, 0.14, 0.15 to about 0.15, 0.16, 0.17, 0.18, 0.19, 0.20, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, or 0.5 wt.% including increments and ranges therein and there between.

### ADDITIVES

### Optional Additives

The compositions can also comprise at least one additive conventionally used in the cosmetics field which does not affect the properties of the compositions according to the invention, such as thickeners, pearlescent agents, preservatives, sunscreens, anionic or nonionic or cationic or amphoteric polymers, proteins, protein hydrolysates, fatty acids, silicones, vegetable, mineral or synthetic oils, gelling agents, fragrances, fillers, screening agents, odor absorbers and coloring materials.

Any one or a combination of additives can be present in the composition according to the invention in proportions which are not limited, but which advantageously fall in the range from 0% to 90% by weight, or 1 to 90% by weight, with respect to the total weight of the cosmetic composition.

### Formulations Comprising the Compositions

Generally, any composition of the invention can be topically applied to the skin (over any cutaneous region of the body) or to the mucous membranes (oral, jugal, gingival, genital, conjunctival, and the like). Depending on the method of administration under consideration, the composition can be provided in any dosage form normally used.

For topical application to the skin, the composition can have the form in particular of aqueous or oily solutions or of dispersions of the lotion or serum type of emulsions with a liquid or semi-liquid consistency of the milk type, obtained by dispersion of a fatty phase in an aqueous phase (O/W) or vice versa (W/O), including those employing silicone and non-silicone oils, or of suspensions or emulsions with a soft consistency of the aqueous gel or cream type, or of microcapsules or microparticles, or of vesicular dispersions of ionic and/or nonionic type or of foams, or they may be provided in the form of toners or micellar water, or they may be biphasic and include water and other solvents such as alcohols and other water-soluble solvents and oils that are not water miscible. Biphasic compositions require pre-application agitation to form an emulsion prior to application to the keratinous tissue, such as skin, which emulsion is of sufficient quality and stability to allow a uniform application of the emulsified phases. Thus, a suitable carrier system according to the disclosure may be selected from any one of oil-in-water emulsions, water -in-oil emulsions, silicone in water emulsions, dispersions, micellar waters, biphasic compositions, and foams. These compositions are prepared according to the usual methods.

The amounts of the various constituents of the compositions according to the invention are those conventionally used in the fields under consideration.

In the cosmetics field, these compositions constitute in particular creams for cleaning, protecting, treating or caring for the face, for the hands, for the feet, for the major anatomical folds or for the body (for example, day creams, night creams, make-up-removing creams, foundation creams or sun creams), liquid foundations, make-up-removing milks, protective or care body milks, sun milks, lotions, gels or foams for caring for the skin, such as cleansing lotions, sun lotions, artificial tanning lotions, bath compositions, deodorizing compositions comprising a bactericidal agent, aftershave gels or lotions, depilatory creams, compositions for countering insect stings or bites, pain-relieving compositions or compositions for treating one or more of aging, sun damaged, oily and acne prone skin, and certain skin diseases, such as eczema, rosacea, psoriasis, lichen and severe pruritus.

### SUITABLE CARRIER

The water and or the glycols in the compositions typically form part or all of a cosmetically acceptable carrier. The cosmetically acceptable carrier can include, for example, glycerin, C1-4 alcohols, organic solvents, fatty alcohols, fatty ethers, fatty esters, polyols, glycols, vegetable oils, mineral oils, liposomes, laminar lipid materials, water, or any combinations thereof. As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

In some instances, cosmetically acceptable carriers may comprise water, a mixture of water and at least one cosmetically acceptable organic solvent, or at least one cosmetically acceptable organic solvent. Additionally, cosmetically acceptable carriers may be or may include ethanol, a glycol ether, for example, dipropylene glycol n-butyl ether, isododecane, mineral oil, propylene glycol, pentylene glycol, hexylene glycol, glycerol, and mixtures thereof.

### Water

In accordance with the various embodiments, water is present in the compositions in a range from about 35% to about 90%, and in some embodiments, from about 35% to about 70%, and in some embodiments, from about 35% to about 65%, and in some embodiments, from about 35% to about 60%, and in some embodiments, about 68% or any suitable combination, sub-combination, range, or sub-range thereof by weight, based on the weight of the composition. One of ordinary skill in the art, however, will appreciate that other ranges are within the scope of the invention. Thus, water is present, by weight, based on the total weight of the composition, from about 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, to about 90 percent, by weight, including increments and ranges therein and there between. One of ordinary skill in the art, however, will appreciate that other ranges are within the scope of the invention.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, that is to say a water comprising trace elements such as zinc, copper, magnesium, etc., reconstituting the characteristics of a thermal water.

The pH of the composition is not limited but is generally between about 2.0 and about 7.0, and in some embodiments, is one of between about 3 and about 4.5. The pH can be adjusted to the desired value by addition of a base (organic or inorganic) to the composition, for example, sodium citrate, ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

The pH of the composition is not limited but is generally not less than 3.5. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, sodium hydroxide, potassium hydroxide or alternatively by addition of an inorganic or organic acid, advantageously a carboxylic acid, such as, for example, citric acid.

In some embodiments, the composition includes one or more solvents comprising alcohol for solubilizing acid, for example, monoalcohols such as monohydric C1-C8 alcohols such as ethanol, propanol, butanol, isopropanol, isobutanol, and benzyl alcohol, and phenylethyl alcohol. In some embodiments, the alcohol solvent is selected from one or a combination of ethanol and isopropyl alcohol. The solvents may also include water-soluble solvents including polyols and short chain mono-alcohols. In some specific embodiments the water-soluble solvents are selected from glycerin, propylene glycol, butylene glycol, and combinations thereof. In one example, a solvent used in accordance with the disclosure is propylene glycol.

In accordance with the various embodiments, the amount of alcohol or glycol present in the composition is in the range from about 5% to about 20%, or from about 6% to about 18%, or from about 10% to about 15%, or any suitable combination, sub-combination, range, or sub-range thereof by weight, based on the weight of the composition. One of ordinary skill in the art, however, will appreciate that other ranges are within the scope of the invention.

Thus, any one of or a combination of alcohols or glycols may be present, by weight, based on the total weight of the composition, from about 5, 6, 7, 8, 9, 10, 11, 12, 13, 14, 15, 16, 17, 18, 19, to about 20 weight percent, including increments and ranges therein and there between.

### OPTIONAL ANTIOXIDANTS

The compositions can also contain one or more antioxidants. Antioxidants can be any antioxidant or other additive suitable for use in cosmetic formulations. Suitable additives include, but are not limited to, resveratrol, tannic acid, polyphenols, amino acids and derivatives thereof, imidazoles, peptides such as carnosine and derivatives, carotenoids, carotenes (such as α-carotene, β-carotene, and lycopene), additional acids selected from α-hydroxy acids (such as citric acid, lactic acid, malic acid, and combinations of these), beta-hydroxy acids (such as propionic acid, beta-hydroybutyric acid, beta-hydroxy beata-methylbutyric acid, carnitine, and combinations of these), tocopherols and derivatives (such as Vitamin E), vitamin A, co-enzyme Q10, bioflavonoids, glutathione, plant extracts (such as rosemary extract, olive leaf extracts, Scutellaria Baicalensis (baicalin), and Mango Leaf Extract (mangiferin), and green tea extracts.

The number of antioxidants present in the compositions can range from about 0.01% to about 20%, or from about 0.1% to about 20%, or from about 0.01% to about 10%, based on the total weight of the composition.

Although the optional additives are given as examples, it will be appreciated that other optional components compatible with cosmetic applications known in the art may be used that are suitable.

In some particular embodiments, the composition may include optional additives selected chosen from terephthalylidene dicamphor sulfonic acid, trisodium ethylenediamine disuccinate, sodium citrate, sodium chloride, hydroxyacetophenone sodium benzoate, potassium sorbate, citric acid, caprylyl glycol, and trisodium ethylenediamine disuccinate, and combinations thereof.

In accordance with the various embodiments, the amount of one or more actives and additives, alone or in combination, when present in the composition according to the disclosure can be present in a range from about 0.001% to about 20%, and in some embodiments, from about 0.05% to about 0.01%, and in some embodiments, from about 0.01% to about 0.1%, and in some embodiments, from about 0.15% to about 5%, and in some embodiments, from about 0.40% to about 4%, and in some embodiments, from about 0.5% to about 2.5%, and in some embodiments, from about 0.1% to about 0.5% and in some embodiments, from about 1% to about 2%, or any suitable combination, sub-combination, range, or sub-range thereof by weight, based on the total weight of the composition. One of ordinary skill in the art, however, will appreciate that other ranges are within the scope of the invention.

### EXAMPLES

Implementation of the present disclosure is provided by way of the following examples. The following examples serve to elucidate aspects of the technology without being limiting in nature.

### Example 1

### Inventive Compositions

TABLE 1

| | Inven- | Inven- | Inven- | Inven- |
|---|---|---|---|---|
| | tive | tive | tive | tive |
| | Exam- | Exam- | Exam- | Exam- |
| INCI US | ple 1 | ple 2 | ple 3 | ple 4 |
| SALICYLIC ACID | 0.1 | 0.2 | 0.1 | 0.1 |
| BENZOIC ACID | 0.2 | 0.1 | 0.2 | 0.15 |
| ZINC PCA | | 0.1 | | |
| ZINC GLUCONATE | | | 0.2 | 0.15 |
| COPPER GLUCONATE | 0.2 | | | |
| | 12.5 | 12.4 | 12.5 | 12.0 |
| CELLULOSE | 1.5 | 1.5 | 1.5 | 1.5 |
| XANTHAN GUM; SODIUM | 1.0 | 1.0 | 1.0 | 0.95 |
| POLYACRYLATE STARCH) | | | | |
| GLYCERYL STEARATE; | 7.5 | 5.0 | 7.5 | 7.5 |
| CETEARYL ALCOHOL (and) | | | | |
| CETEARYL GLUCOSIDE | | | | |
| GLYCOLS | 13.0 | 13.0 | 13.0 | 13.0 |
| WATER | 61.2 | 63.9 | 61.4 | 62.5 |
| VITAMIN | 0.1 | 0.4 | 0.1 | 0.1 |
| ACTIVE COMPOUND | 0.04 | 0.04 | 0.04 | 0.04 |
| ESSENTIAL OILS | 0.2 | 0.2 | 0.2 | 0.11 |
| VEGETAL EXTRACTS | 2.3 | 2.0 | 2.0 | 0.0 |

### Example 2

### Evaluation of stability and Microbial Challenge Test

Inventive and Comparative compositions were evaluated for stability and Microbial Challenge Tests. The Challenge test protocol similar to the PCPC guidelines was followed to assess efficacy against a broad spectrum of microorganisms.

### Results

As shown by the results in Table 2, the use of salicylic acid and benzoic acid as preservatives alone were not strong enough to reduce the E. faecalis (gram-positive bacteria) within the formulation. Typical preservatives for the required log reduction in gram-positive bacteria include chlorphenesin and phenoxyethanol; however, these are petrochemical-based preservatives and do not align with the need for a highly natural formula. Alcohol and capryloyl salicylic acid are also beneficial for preservation;

however, an alcohol-free formula was desired and capryloyl salicylic acid can cause instabilities within formulations despite low levels.

The addition of transition metal 2+ions such as copper and zinc to provide additional microbial preservation against gram-positive bacteria were tested. When copper gluconate was added to the composition, a blue-green tint was observed. Such color was undesirable for the formulation. This was addressed by using an alternative transition metal 2+ion, i.e. zinc, which is colorless. Zinc salts are typically used for their anti-acneic and anti-seborrhea properties. These properties inspired trials with the inclusion of zinc gluconate and zinc pca by the formulation needing additional strength for gram-positive bacteria since bacteria are common causes for acne. Gluconates also are beneficial for hydration by being skin-conditioning agents, which led to focus on zinc gluconate as an additive for preservation for the instant disclosure.

The goal of the instant disclosure was to provide a preservation for non-protective packaging, such as a jar, for highly natural formulas without using alcohol or petrochemical-based preservatives and provides a stable formulation for a highly sensitive composition.

TABLE 2
Stability and Challenge test results

| | | Inv. | Inv. | Inv. | Inv. | Comp. | Comp. | Comp. | Comp. |
|---|---|---|---|---|---|---|---|---|---|
| | INCI US | Ex. 1 | Ex. 2 | Ex. 3 | Ex. 4 | Ex. 1 | Ex. 2 | Ex. 3 | Ex. 4 |
| TESTS | SALICYLIC ACID | 0.1 | 0.15 | 0.1 | 0.1 | 0.3 | 0.15 | 0.15 | 0.1 |
| | BENZOIC ACID | 0.15 | 0.1 | 0.15 | 0.15 | | 0.1 | 0.15 | |
| | COPPER GLUCONATE | 0.15 | | | | 0.15 | | | |
| | ZINC PCA | | 0.1 | | | | | | |
| | ZINC GLUCONATE | | | 0.15 | 0.15 | | | | |
| | CAPRYLOYL | | | | | | | | |
| | SALICYLIC ACID | | | | | | | | |
| | PHENOXYETHANOL | | | | | | | | |
| | CHLORPHENESIN | | | | | | | | |
| | ALCOHOL DENAT. | | | | | | | | |
| | FATTY COMPOUND | 12.48 | 12.38 | 12.48 | 12.48 | 12.48 | 12.38 | 12.38 | 14 |
| | CELLULOSE | 1.5 | 1.5 | 1.5 | 1.5 | 1.5 | 1.5 | 1.5 | 1.5 |
| | POLYMER | 0.95 | 0.95 | 1.05 | 0.95 | 0.95 | 0.95 | 0.95 | 0.95 |
| | SURFACTANT | 7.5 | 5 | 7.5 | 7.5 | 7.5 | 7.5 | 5 | 7.5 |
| | SOLVENT | 13.3 | 13.3 | 13.3 | 13.3 | 13.3 | 13.3 | 15.3 | 13.3 |
| | WATER | 61.2 | 63.9 | 61.4 | 62.5 | 61.1 | 61.1 | 61.6 | 58.6 |
| | VEGETAL EXTRACT | 2.33 | 2 | 2 | 1 | 2.33 | 2.3 | 2.3 | 3.3 |
| | VITAMIN | 0.1 | 0.4 | 0.1 | 0.1 | 0.1 | 0.4 | 0.4 | 0.1 |
| | ACTIVE COMPOUND | 0.04 | 0.04 | 0.04 | 0.04 | 0.04 | 0.04 | 0.04 | 0.44 |
| | ESSENTIAL OILS | 0.20 | 0.22 | 0.21 | 0.22 | 0.20 | 0.20 | 0.20 | 0.20 |
| | DYE/PIGMENT | 0 | 0 | 0 | 0.03 | 0 | 0.03 | 0.03 | 0 |
| Stability | | STABLE | STABLE | STABLE | STABLE | N/A | N/A | N/A | N/A |
| Microbial | | Pass | Pass | Pass | Pass | Borderline | Fail | Fail | Failing |
| Challenge | | | | | | | | | |
| Testing | | | | | | | | | |

| | | Comp. | Comp. | Comp. | Comp. | Comp. | Comp. | Comp. |
|---|---|---|---|---|---|---|---|---|
| | INCI US | Ex. 5 | Ex. 6 | Ex. 7 | Ex. 8 | Ex. 9 | Ex. 10 | Ex. 11 |
| TESTS | SALICYLIC ACID | 0.1 | 0.1 | 0.15 | 0.1 | 0.15 | 0.1 | 0.1 |
| | BENZOIC ACID | 0.15 | 0.15 | 0.1 | 0.15 | 0.1 | 0.15 | |
| | COPPER GLUCONATE | | | | | | | |
| | ZINC PCA | | | | | | | |
| | ZINC GLUCONATE | | | | | | 0.15 | 0.15 |
| | CAPRYLOYL | | | | | | 0.15 | 0.3 |
| | SALICYLIC ACID | | | | | | | |
| | PHENOXYETHANOL | | | | | 0.5 | | |
| | CHLORPHENESIN | | 0.27 | 0.2 | 0.1 | | | |
| | ALCOHOL DENAT. | 3.5 | | | | | | |
| | FATTY COMPOUND | 12.48 | 12.48 | 12.38 | 12.48 | 12.38 | 12.48 | 12.48 |
| | CELLULOSE | 1.5 | 1.5 | 1.5 | 1.5 | 1.5 | 1.5 | 1.5 |
| | POLYMER | 0.95 | 0.95 | 0.95 | 0.95 | 0.95 | 1.05 | 1.05 |
| | SURFACTANT | 7.5 | 7.5 | 5 | 7.5 | 5 | 7.5 | 7.5 |
| | SOLVENT | 13.3 | 13.3 | 13.3 | 13.3 | 13.3 | 13.3 | 13.3 |
| | WATER | 58.2 | 61.1 | 63.8 | 61.6 | 63.1 | 61.3 | 61.3 |
| | VEGETAL EXTRACT | 2 | 2.3 | 2 | 2 | 2.3 | 2 | 2 |
| | VITAMIN | 0.1 | 0.1 | 0.4 | 0.1 | 0.4 | 0.1 | 0.1 |
| | ACTIVE COMPOUND | 0.04 | 0.04 | 0.04 | 0.04 | 0.04 | 0.04 | 0.04 |
| | ESSENTIAL OILS | 0.21 | 0.20 | 0.22 | 0.21 | 0.20 | 0.21 | 0.21 |
| | DYE/PIGMENT | 0 | 0 | 0 | 0 | 0.03 | 0 | 0 |
| Stability | | separation | undesirable | undesirable | undesirable | unstable | UNSTABLE | UNSTABLE |
| | | of emulsion | texture | texture | texture | | | |
| Microbial | | Pass | Pass | Pass | Pass | N/A | N/A | N/A |
| Challenge | | | | | | | | |
| Testing | | | | | | | | |

Stable compositions were validated by Inv. Ex. 1, Inv. Ex. 2, Inv. Ex. 3 and Inv. Ex.4.These compositions contained the preservative system mentioned in the instant disclosure. These Inventive Examples passed the microbiological tests as it is shown in Table 2.

Different preservative systems were tested as well. For Examples, Comp. Ex. 1, Comp. Ex. 2, Comp. Ex. 3 and Comp. Ex 4 were prepared similarly to the inventive examples except that one or two of the ingredients of the preservative systems were not present. It was observed that if only salicylic acid and benzoic acid were present, the microbiological tests failed (i.e. Comp. Ex. 2, Comp. Ex. 3 and Comp. Ex.4). In the case of Comp. Ex. 1 where the preservative system contained salicylic acid and copper gluconate, the microbiological test was borderline and consequently not acceptable.

Other comparatives examples were tested without divalent metal salts (i.e. Comp. Ex. 5, Comp. Ex. 6, Comp. Ex.7, Comp. Ex. 8, Comp. Ex. 9). Even though some passed the microbiological tests (i.e. Comp. Ex. 5, Comp. Ex. 6, Comp. Ex.7, Comp. Ex. 8), the compositions were not stable and/or presented an undesirable texture. These results demonstrated the criticality of the preservative system. For example, in Comp. Ex. 5, the preservative system of the composition contained 0.1% of Salicylic Acid and 0.15% of benzoic acid with 3.5% of alcohol denatured, despite a good microbiological test, the composition was not stable and a separation of the emulsion was observed.

In the case of Comp. Ex. 10 and Comp. Ex. 11, the use of capryloyl salicylic acid resulted in an unstable formula with separation of the emulsion.

In the case of Comp. Ex. 5, the use of a denatured alcohol provided a non-protective packaging pass for microbial testing, however, resulted in undesirable aesthetics by having a glossy surface which appeared to be leading toward separation of the emulsion after 2 weeks at 45C.

In the case of Comp. Ex. 6, Comp. Ex. 7 and Comp. Ex. 8, the use of chlorphenesin (a known non-natural preservative) provided a non-protective packaging pass for microbial challenge testing, however, the use of this material was undesirable due to the formulation's high natural content and the loss of mousse-like texture and glossy surface appearance after 2 weeks in stability. Furthermore, Chlorphenesin is also a petrochemical-derived preservative which was undesirable for the formula.

In the case of Comp. Ex. 9., the use of non-natural phenoxyethanol resulted in an unstable formulation through separation of the emulsion.

As shown by the results in Table 2, the inventive examples demonstrated the microbial preservation against gram-positive bacteria of the preservative system constituted by the association of salicylic acid, benzoic acid and transition metal 2+ ions such as copper and zinc.

While the disclosure has been described with reference to described embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiment disclosed as the best mode contemplated for carrying out this disclosure, but that the disclosure will include all embodiments falling within the scope of the appended claims.

The articles “a” and “an,” as used herein, mean one or more when applied to any feature in embodiments of the present disclosure described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is

“At least one,” as used herein, means one or more and thus includes individual components as well as mixtures/combinations.

The transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinarily associated with the specified material(s). The term “consisting essentially of” limits the scope of a claim to the specified elements, steps or material(s) and those that do not materially affect the basic and novel characteristic(s) of the claimed disclosure. All materials and methods described herein that embody the present disclosure can, in alternate embodiments, be more specifically defined by any of the transitional terms “comprising,” “consisting essentially of,” and “consisting of.”

The terms “free” and “devoid” indicates that no reliably measurable excluded material is present in the composition, typically 0% by weight, based on the total weight of the composition. The term “essentially free” means that, while it prefers that no excluded material is present in the composition, it is possible to have very small amounts of the excluded material in the composition of the invention, provided that these amounts do not materially affect the advantageous properties of the composition. In particular, “essentially free” means that excluded material can be present in the composition at an amount of less than about 0.1% by weight, based on the total weight of the composition.

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

All percentages and ratios are calculated by weight unless otherwise indicated. All percentages are calculated based on the total composition unless otherwise indicated. Generally, unless otherwise expressly stated herein, “weight” or “amount” as used herein with respect to the percent amount of an ingredient refers to the amount of the raw material comprising the ingredient, wherein the raw material may be described herein to comprise less than and up to 100% activity of the ingredient. Therefore, weight percent of an active in a composition is represented as the amount of raw material containing the active that is used, and may or may not reflect the final percentage of the active, wherein the final percentage of the active is dependent on the weight percent of active in the raw material.

The terms “weight percent” and “wt%” may be used interchangeably and mean percent by weight, based on the total weight of a composition, article or material, except as may be specified with respect to, for example, a phase, or a system that is a component of a composition, article or material. All ranges and amounts given herein are intended to include subranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. Further, it is understood that when an amount of a component is given, it is intended to signify the amount of the active material unless otherwise specifically stated.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A preservative system comprising:
a. salicylic acid;
b. weak organic acid or its salts;
c. one or more divalent transition metal salts chosen from salts of zinc, nickel, copper, silver, manganese, cobalt, and combinations thereof; and
wherein all percentages by weight are based on the total weight of the preservative system.

2. The preservative system of claim 1, wherein the weak organic acid or its salts is chosen from benzoic acid, sorbic acid, levulinic acid, anisic acid, dehydroacetic acid, propionic acid and combinations thereof.

3. The preservative system of claim 2, wherein the weak organic acid or its salts is benzoic acid or its salts.

4. The preservative system of claim 1, wherein the one or more divalent transition metal salts are chosen from a divalent transition metal salt of an organic acid, a divalent transition metal salt of an inorganic acid, and combinations thereof.

5. The preservative system of claim 1, wherein the one or more divalent metal salts include divalent metal salts of an organic acid chosen from zinc lactate, zinc gluconate, zinc phenolsulfonate, zinc salicylate, zinc pyrrolidone carboxylic acid (Zinc PCA), zinc citrate, zinc ascorbate, copper gluconate, copper sulfate, their salt derivatives, and combinations thereof.

6. The preservative system of claim 1, wherein the one or more divalent metal salts include divalent metal salts of an inorganic acid chosen from metal halides, metal hydroxides, metal sulfates, metal oxides, and combinations thereof.

7. The preservative system of claim 4, wherein the one or more divalent metal salts of an organic acid includes a zinc salt.

8. The preservative system of claim 7, wherein the zinc salt is zinc gluconate.

9. A cosmetic composition comprising the preservative system of claim 1, a suitable carrier system, and additives selected from fatty compounds, fillers, polymers, surfactants, solvents, essential oils, vegetal extract, conditioners, emollients, skin caring ingredients, thickeners, anti-oxidants, other preservatives, active ingredients, fragrances and combination thereof.

10. The cosmetic composition of claim 9, wherein the one or more divalent transition metal salts is present from about 0.01 to about 0.5 wt.% based on the total weight of the cosmetic composition.

11. The cosmetic composition of claim 9, wherein the salicylic acid is present from about 0.01 to about 0.5 wt.% based on the total weight of the cosmetic composition.

12. The cosmetic composition of claim 9, wherein the benzoic acid or its salts is present from about 0.01 to about 0.3 wt.% based on the total weight of the cosmetic composition.

13. The cosmetic composition of claim 9, wherein the cosmetic composition is in the form of an oil-in-water emulsion.

14. The cosmetic composition of claim 9, wherein the cosmetic composition is in the form of a water-in-oil emulsion.

15. The preservative system of claim 1 that can be used in hair compositions, skin care compositions, cleansing compositions, makeup compositions.

16. A cosmetic composition comprising:
A) A preservative system comprising:
a. from about 0.01 to about 0.5 wt.% of salicylic acid;
b. from about 0.01 to about 0.3 wt.% of benzoic acid or its salts;
c. from about 0.01 to about 0.5 wt.% of one or more divalent transition metal salts;
B) from about 0.1 to about 15 wt.% of one or more fatty compound;
C) from about 0.1 to about 5 wt.% of one or more fillers;
D) from about 0.1 to about 10 wt.% of one or more surfactants; and
E) a suitable carrier;
wherein all percentages by weight are based on the total weight of the cosmetic composition.

17. A method of preserving a cosmetic composition by incorporating the preservative system of claim 1.
